(19) United States
(12) Patent Application Publication
(10) Pub. No.: US 2016/0243224 A1
(43) Pub. Date: Aug. 25, 2016

(54) TWEAK ANTAGONISTS FOR TREATING LUPUS NEPHRITIS AND MUSCLE ATROPHY

(71) Applicant: BIOGEN MA INC., Cambridge, MA (US)

(72) Inventors: Nicolas Wisniacki, London (GB); Linda Burkly, Monument Beach, MA (US); Gerald Russell Galluppi, Creve Coeur, MO (US); Ivan Alexandrov Nestorov, Acton, MA (US)

(21) Appl. No.: 15/026,394

(22) PCT Filed: Oct. 3, 2014
(86) PCT No.: PCT/US14/59007

§ 371 Date: Mar. 31, 2016

Related U.S. Application Data
(60) Provisional application No. 61/887,209, filed on Oct. 4, 2013.

(51) Int. Cl. A61K 39/395; A61K 45/06; C07K 16/28

(52) U.S. Cl. CPC A61K 39/3955; C07K 16/2875; A61K 45/06; C07K 2317/565; C07K 2317/51; C07K 2317/515

(57) ABSTRACT

Methods and compositions for treating lupus nephritis and muscle atrophy with anti-TWEAK antibodies are provided. Methods that include administering therapeutically effective amounts of anti-TWEAK antibodies to human subjects already receiving a standard treatment for lupus nephritis are also encompassed. Particularly useful dosages are provided.

### CROSS-REFERENCE TO RELATED APPLICATIONS

This application claims the benefit of U.S. Provisional Application No. 61/887,209, filed Oct. 4, 2013, the contents of which are hereby incorporated by reference in its entirety.

### BACKGROUND

TWEAK is a proinflammatory cytokine belonging to the TNF ligand superfamily. TWEAK mediates its biological activity through its receptor Fn14, which is expressed by different tissue cell types including mesenchymal, epithelial, and endothelial cells. TWEAK-Fn14 signaling can lead to multiple cellular responses, including production of proinflammatory cytokines, chemokines, and matrix metalloproteinases. Activation of the TWEAK-Fn14 pathway is tightly regulated in vivo, where highly induced expression of TWEAK and Fn14 occurs locally in the specific organs affected by tissue injury and inflammatory disease. Therein, TWEAK acts on resident tissue cell types through its receptor Fn14, mediating multiple cellular responses that contribute to pathological tissue damage and remodeling.

### SUMMARY

In one aspect, methods for treating lupus nephritis are provided. In certain embodiments, the method comprises administering a therapeutically effective amount of an anti-TWEAK (TNF-like weak inducer of apoptosis) antibody to a subject having or suspected of having lupus nephritis. In some embodiments, the therapeutically effective amount of the anti-TWEAK antibody is 20 mg/kg. In some embodiments, the therapeutically effective amount of the anti-TWEAK antibody is 3 mg/kg.

In some embodiments, the method for treating lupus nephritis comprises the step of administering a therapeutically effective amount of an anti-TWEAK antibody together with one or more non-TWEAK-related agents. The non-TWEAK-related agent may be provided prior to, concurrently with, or after the anti-TWEAK antibody. The subject may have been receiving the non-TWEAK related agent prior to receiving the anti-TWEAK antibody, and the subject may or may not continue to receive the same non-TWEAK-related agent after initiation of the anti-TWEAK antibody. The non-TWEAK-related agent may be, for example, a steroid, such as prednisone, and/or an immunosuppressant such as mycophenolate mofetil (MMF). In one embodiment, administration of anti-TWEAK allows for administration of a reduced amount of the non-TWEAK-related agent and can reduce the presence of side effects.

In some embodiments, the method for treating lupus nephritis comprises the step of administering an anti-TWEAK antibody and a steroid to a subject having or suspected of having lupus nephritis. In some embodiments, the method for treating lupus nephritis comprises the step of administering an anti-TWEAK antibody and an immunosuppressant (e.g., MMF) to a subject having or suspected of having lupus nephritis. In still other embodiments, the method for treating lupus nephritis comprises administering an anti-TWEAK antibody, a steroid, and an immunosuppressant (e.g., MMF) to a subject having or suspected of having lupus nephritis.

In some embodiments, the method for treating lupus nephritis comprises administering 20 mg/kg of the anti-TWEAK antibody described herein to a subject already receiving a steroid. Methods for treating lupus nephritis comprising administering 20 mg/kg of an anti-TWEAK antibody described herein to a subject already receiving a steroid and an immunosuppressant (e.g., MMF) are also encompassed. In these embodiments, the subject may or may not continue to receive the steroid and/or an immunosuppressant (e.g., MMF) during treatment with the anti-TWEAK antibody. In one embodiment, the amount of the second agent administered can be decreased during or after treatment with anti-TWEAK antibody.

In some embodiments, the method for treating lupus nephritis comprises administering 3 mg/kg of the anti-TWEAK antibody described herein to a subject already receiving a steroid. Methods for treating lupus nephritis comprising administering 3 mg/kg of an anti-TWEAK antibody described herein to a subject already receiving a steroid and an immunosuppressant (e.g., MMF) are also encompassed. In these embodiments, the subject may or may not continue to receive the steroid and/or an immunosuppressant (e.g., MMF) during treatment with the anti-TWEAK antibody. In one embodiment, the amount of the second agent administered can be decreased during or after treatment with anti-TWEAK antibody.

In another aspect, compositions for treating lupus nephritis are provided. Compositions comprising therapeutically effective amounts of an anti-TWEAK antibody are encompassed, as are compositions comprising an anti-TWEAK antibody and a steroid, an anti-TWEAK antibody and an immunosuppressant, and an anti-TWEAK antibody, a steroid, and an immunosuppressant. Use of these compositions to treat lupus nephritis is fully encompassed. In certain embodiments, the composition comprises an amount of antibody appropriate for administration of 20 mg/kg of an anti-TWEAK antibody, 3 mg/kg of an anti-TWEAK antibody, 20 mg/kg of an anti-TWEAK antibody and a steroid, 20 mg/kg of an anti-TWEAK antibody, a steroid, and an immunosuppressant (e.g., MMF), 3 mg/kg of an anti-TWEAK antibody and a steroid, or 3 mg/kg of an anti-TWEAK antibody, a steroid, and an immunosuppressant (e.g., MMF). A composition comprising an amount of an anti-TWEAK antibody appropriate for administration of 20 mg/kg of the antibody optionally comprises a fixed dose of 1,600 mg of the antibody. A composition comprising an amount of an anti-TWEAK antibody appropriate for administration of 3 mg/kg of the antibody optionally comprises a fixed dose of 240 mg of the antibody. The compositions may be formulated for separate or concurrent administration. The disclosure encompasses any of the compositions described herein for use in the treatment of lupus nephritis.

In another aspect, this disclosure provides methods and compositions for treating muscle atrophy. In certain embodiments, the method involves administering a therapeutically effective amount of an anti-TWEAK antibody to subject having or suspected of having muscle atrophy. In some embodiments, the therapeutically effective amount of the anti-TWEAK antibody is 20 mg/kg.

In some embodiments, the method for treating muscle atrophy comprises the step of administering a therapeutically effective amount of an anti-TWEAK antibody together with one or more non-TWEAK-related agents. The non-TWEAK-related agent may be provided prior to, concurrently with, or after the anti-TWEAK antibody. The subject may have been receiving the non-TWEAK related agent prior to receiving the anti-TWEAK antibody, and the subject may or may not continue to receive the same non-TWEAK-related agent after initiation of the anti-TWEAK antibody. In one embodiment, the non-TWEAK-related agent may be, for example, a branched amino acid such as leucine, isoleucine, valine, or lysine, as part of an amino acid therapy. In one embodiment, the non-TWEAK-related agent may be, for example, a selective androgen receptor modulator (SARM) such as tamoxifen, enobosarm, BMS-564,929, LGD-4033, AC-262,356, JNJ-28330835, LGD-2226, LGD-3303, S-40503, or S-23. In another embodiment, the non-TWEAK-related agent may be, for example, a low molecular weight heparin (LMWH) such as enoxaparin. In another embodiment, the non-TWEAK-related agent may be, for example, an agent that induces hypertrophy, such as a myostatin pathway inhibitor. In one embodiment, administration of anti-TWEAK allows for administration of a reduced amount of the non-TWEAK-related agent and can reduce the presence of side effects.

In some embodiments, the method for treating muscle atrophy comprises the step of administering an anti-TWEAK antibody and an amino acid (e.g., leucine, isoleucine, valine, or lysine) to a human subject having or suspected of having muscle atrophy. In some embodiments, the method for treating muscle atrophy comprises the step of administering an anti-TWEAK antibody and a SARM to a human subject having or suspected of having muscle atrophy. In certain embodiments, the method for treating muscle atrophy comprises the step of administering an anti-TWEAK antibody and a LMWH (e.g., enoxaparin) to a human subject having or suspected of having muscle atrophy. In certain embodiments, the method for treating muscle atrophy comprises the step of administering an anti-TWEAK antibody and an agent that induces hypertrophy (e.g., a myostatin pathway inhibitor) to a human subject having or suspected of having muscle atrophy. In still other embodiments, the method for treating muscle atrophy comprises administering an anti-TWEAK antibody, an amino acid, a SARM and/or a LMWH to a human subject having or suspected of having muscle atrophy.

In certain embodiments, the human subject being treated for muscle atrophy has this condition as a result of a co-morbidity of a disease such as cancer, acquired immunodeficiency syndrome (AIDS), congestive heart failure, chronic obstructive pulmonary disease (COPD), renal failure, liver disease, cachexia, alcohol-associated myopathy, amyotrophic lateral sclerosis (ALS), dermatomyositis, polymyositis, Guillain-Barre syndrome, motor neuropathy, muscular dystrophy, glycogen storage disease, mitochondrial myopathy, lipid myopathy, central tubular myopathy, rhabdomyolysis, alcoholic myopathy, inflammatory myopathy, glucocorticoid-induced myopathy, osteoarthritis, rheumatoid arthritis, spinal cord injury, stroke, inclusion body myositis, myotonic dystrophy, sarcopenia, diaphragm atrophy (e.g., resulting from intensive care unit stay), or other muscle atrophy resulting from intensive care unit stay.

In some embodiments, the human subject being treated with an anti-TWEAK antibody or antigen binding fragment thereof has, is suspected of having, or is at risk of developing disuse muscle atrophy. In other embodiments, the human subject being treated with an anti-TWEAK antibody or antigen binding fragment thereof has, is suspected of having, or is at risk of developing neurogenic atrophy.

In some embodiments, the human subject being treated with an anti-TWEAK antibody or antigen binding fragment thereof has or is at risk of developing muscle atrophy due to immobilization. In certain embodiments, the human subject being treated with an anti-TWEAK antibody or antigen binding fragment thereof has or is at risk of developing muscle atrophy due to malnutrition. In other embodiments, the human subject being treated with an anti-TWEAK antibody or antigen binding fragment thereof has or is at risk of developing muscle atrophy due to long-term corticosteroid therapy. In some embodiments, the human subject being treated with an anti-TWEAK antibody or antigen binding fragment thereof has or is at risk of developing muscle atrophy due to a burn.

In certain embodiments, the human subject being treated for muscle atrophy is administered an anti-TWEAK antibody or antigen-binding fragment thereof at a dose of 20 mg/kg every 4 weeks. In some embodiments, the human subject is administered a dose of 20 mg/kg every 3 weeks. In certain embodiments, the human subject is administered a dose of 20 mg/kg every 2 weeks. In one embodiments, the human subject is administered a dose of 20 mg/kg every week.

In some embodiments, the human subject being treated for muscle atrophy is administered eight doses, seven doses, six doses, five doses, four doses, three doses, two doses, or one dose of an anti-TWEAK antibody or antigen-binding fragment thereof, wherein each dose is 20 mg/kg.

In some embodiments, the human subject is administered the anti-TWEAK antibody or antigen-binding fragment thereof intravenously.

In another aspect, this disclosure provides a composition comprising an amount of antibody appropriate for administration of 20 mg/kg of an anti-TWEAK antibody and one or more of a SARM, a branched amino acid, a low molecular weight heparin, or an agent that induces hypertrophy (e.g., a myostatin pathway inhibitor). A composition comprising an amount of an anti-TWEAK antibody appropriate for administration of 20 mg/kg of the antibody optionally comprises a fixed dose of 1,600 mg of the antibody. In certain embodiments, the composition is a pharmaceutical composition that includes a pharmaceutically acceptable carrier.

For each embodiment described above, the anti-TWEAK antibody or antigen-binding fragment thereof comprises a heavy chain variable domain (VH) complementarity determining region (CDR) 1 comprising the amino acid sequence set forth in SEQ ID NO:4, a VH CDR2 comprising the amino acid sequence set forth in SEQ ID NO:5, and a VH CDR3 comprising the amino acid sequence set forth in SEQ ID NO:6 or 7; and a light chain variable domain (VL) CDR1 comprising the amino acid sequence set forth in SEQ ID NO:11 or 12, a VL CDR2 comprising the amino acid sequence set forth in SEQ ID NO:13 or 14, and a VL CDR3 comprising the amino acid sequence set forth in SEQ ID NO:15 or 16.

In one embodiment, the anti-TWEAK antibody or antigen-binding fragment thereof comprises a VH CDR1 comprising the amino acid sequence set forth in SEQ ID NO:1, a VH CDR2 comprising the amino acid sequence set forth in SEQ ID NO:2, and a VH CDR3 comprising the amino acid sequence set forth in SEQ ID NO:3; and a VL CDR1 comprising the amino acid sequence set forth in SEQ ID NO:8, a VL CDR2 comprising the amino acid sequence set forth in SEQ ID NO:9, and a VL CDR3 comprising the amino acid sequence set forth in SEQ ID NO:10.

In another embodiment, the anti-TWEAK antibody or antigen-binding fragment thereof comprises a heavy chain variable domain comprising the amino acid sequence set forth in SEQ ID NO:17, and a light chain variable domain comprising the amino acid sequence set forth in SEQ ID NO:18 or 19, or an antigen binding fragment thereof.

In one embodiment, the anti-TWEAK antibody comprises a heavy chain comprising the amino acid sequence set forth in SEQ ID NO:20, and a light chain comprising the amino acid sequence set forth in SEQ ID NO:21.

For each embodiment described above, the method may further comprise evaluating the subject for levels of soluble TWEAK, or analyzing the results of a test that evaluated the subject for levels of soluble TWEAK. In some embodiments, the subject is administered any of the compositions described herein if the level of soluble TWEAK is increased as compared to a healthy control.

In one embodiment, the evaluation includes contacting a biological sample of the subject, preferably a urine, serum, plasma, CSF or synovial fluid sample, with an agent that detects TWEAK, a TWEAK receptor (TWEAK-R) or a biomarker whose expression is modulated (e.g., increased) by TWEAK (e.g., in mesangial cells). In other embodiments, the method comprises analyzing the results of a test that evaluates the subject for levels of soluble TWEAK, and administering a therapeutically effective amount of an anti-TWEAK antibody to the patient if the levels of soluble TWEAK are determined to be increased compared to a healthy control. In this embodiment, the therapeutically effective amount of the anti-TWEAK antibody for treatment of lupus nephritis is selected from 20 mg/kg and 3 mg/kg. In this embodiment, the therapeutically effective amount of the anti-TWEAK antibody for treatment of muscle atrophy is 20 mg/kg.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting. Other features and advantages of the invention will be apparent from the following detailed description, and from the claims.

### DETAILED DESCRIPTION

### Lupus Nephritis

Lupus nephritis (LN) remains a major cause of morbidity and mortality in SLE patients. Overt renal disease is found in at least one-third to one-half of SLE patients, with reports of 5-year renal survival with treatment ranging from 46-95%.

The classification of LN has varied over the years, and is well documented. The World Health Organization (WHO) published an initial classification in 1982, and a revised classification in 1995. See, Weening et al. (2004) J Am Soc Nephrol 15:241-250 at Table 2, page 246, and cited references 1 and 2. A third classification was proposed in 2003, and is referred to by those of skill in the art as the “International Society of Nephrology/Renal Pathology Society (ISN/RPS) 2003 classification of lupus nephritis,” or “ISN/RPS 2003” for short. Id. at 247. See, also, Table 1.

TABLE 1
ISN/RPS 2003 classification of lupus nephritis

Cass I Minimal mesangial lupus nephritis
Normal glomeruli by light microscopy, but mesangial immune deposits by immunofluorescence
Class II Mesangial proliferative lupus nephritis
Purely mesangial hypercellularity of any degree or mesangial matrix expansion by light
microscopy, with mesangial immune deposits
May be a few isolated subepithelial or subendothelial deposits visible by immunofluorescence or
electron microscopy, but not by light microscopy
Class III Focal lupus nephritis^{a}
Active or inactive focal, segmental or global endo- or extracapillary glomerulonephritis involving
<50% of all glomeruli, typically with focal subendothelial immune deposits, with or without
mesangial alterations
Class III (A) Active lesions: focal proliferative lupus nephritis
Class III (A/C) Active and chronic lesions: focal proliferative and sclerosing lupus nephritis
Class III (C) Chronic inactive lesions with glomerular scars: focal sclerosing lupus nephritis
Class IV Diffuse lupus nephritis^{b}
Active or inactive diffuse, segmental or global endo- or extracapillary glomerulonephritis
involving >50% of all glomeruli, typically with diffuse subendothelial immune deposits, with or
without mesangial alterations. This class is divided into diffuse segmental(IV-S) lupus nephritis
when >50% of the involved glomeruli have segmental lesions, and diffuse global (IV-G) lupus
nephritis when >50% of the involved glomeruli have global lesions. Segmental is defined as a
glomerular lesion that involves less than half of the glomerular tuft. This class includes cases
with diffuse wire loop deposits but with little or no glomerular proliferation
Class IV-S (A) Active lesions: diffuse segmental proliferative lupus nephritis
Class IV-G (A) Active lesions: diffuse global proliferative lupus nephritis
Class IV-S (A/C)
Active and chronic lesions: diffuse segmental proliferative and sclerosing lupus nephritis
Active and chronic lesions: diffuse global proliferative and sclerosing lupus nephritis
Class IV-S (C) Chronic inactive lesions with scars: diffuse segmental sclerosing lupus
nephritis
Class IV-G (C) Chronic inactive lesions with scars: diffuse global sclerosing lupus nephritis
Class V Membranous lupus nephritis
Global or segmental subepithelial immune deposits or their morphologic sequelae by light
microscopy and by immunofluorescence or electron microscopy, with or without mesangial
alterations
Class V lupus nephritis may occur in combination with class III or IV in which case both will be
diagnosed
Class V lupus nephritis show advanced sclerosis
Class VI Advanced sclerosis lupus nephritis
> or equal to 90% of glomeruli globally sclerosed without residual activity
^{a}Physician would indicate the proportion of glomeruli with active and with sclerotic lesions.
^{b}Physician would indicate and grades the proportion of glomeruli with fibrinoid necrosis and/or cellular crescents.
In each physician would indicate and grade (mild, moderate, severe) tubular atrophy, interstitial inflammation and fibrosis, severity of arteriosclerosis or other vascular lesions.

In one embodiment, subjects meeting one or more of the criteria in Table 1 above are treated using the subject methods. Specific patient populations are described in more detail below.

### Determination of Patient Population for Treatment of Lupus Nephritis

In some embodiments, the human subject who has, or is suspected of having, LN has the following characteristics: 1) the subject has been diagnosed with SLE; 2) the subject has been diagnosed with ISN/RPS 2003 Class III or IV LN; and 3) the subject has proteinuria defined as urine protein/creatinine ratio (uPCR) greater than 1.0. Subjects that meet these criteria may be administered therapeutically effective amounts of an anti-TWEAK antibody described herein, or therapeutically effective amounts of an anti-TWEAK antibody described herein in combination with a steroid and/or an immunosuppressant.

In other embodiments, the human subject who has, or is suspected of having, LN has the following characteristics: 1) a biopsy-proven Class III or IV LN as classified by the ISN/RPS 2003; and 2) a uPCR greater than 1.

In further embodiments, the human subject to be treated by the methods described herein has a diagnosis of SLE, and a soluble TWEAK level that is increased relative to a healthy control. Measurement of soluble TWEAK levels is described herein, and also in WO 2006/138219, at, for example, pages 4-6, and 36.

In certain embodiments, the human subject will not be treated according to the methods described herein if 12 or more weeks after diagnosis with LN, they have a greater than or equal to 30% increase in serum creatinine, optionally measured by two successive measurements separated by greater than or equal to 4 weeks, as compared to baseline, and have creatinine values outside normal range.

In some embodiments, a diagnosis of SLE is made when at least four of the 11 criteria for classification of SLE are met. See, Hochberg M C (1997) Updating the American College of Rheumatology revised criteria for the classification of systemic lupus erythematosus [letter]. Arthritis Rheum 40:1725. The criteria used in diagnosing SLE are shown in Table 2 below. In some embodiments, a diagnosis of SLE is made when at least four of the 11 criteria for classification of SLE are met, wherein at least one of the criteria is a positive antinuclear antibody (ANA), anti-SM, or anti-dsDNA antibody.

TABLE 2
Hochberg criteria for classification of SLE (1997)

| 1. Malar Rash | Fixed erythema, flat or raised, over the malar eminences, tending to | |
|---|---|---|
| | spare the nasolabial folds | |
| 2. Discoid rash | Erythematous raised patches with adherent keratotic scaling and | |
| | follicular plugging; atrophic scarring may occur in older lesions | |
| 3. Photosensitivity | Skin rash as a result of unusual reaction to sunlight, by patient history | |
| | or physician observation | |
| 4. Oral ulcers | Oral or nasopharyngeal ulceration, usually painless, observed by | |
| | physician | |
| 5. Nonerosive | Involving 2 or more peripheral joints, characterized by tenderness, | |
| arthritis | swelling, or effusion | |
| 6. Pleuritis or | 1. | Pleuritis--convincing history of pleuritic pain or rubbing heard |
| pericarditis | | by a physician or evidence of pleural effusion |
| | | OR |
| | 2. | Pericarditis--documented by electrocardigram or rub or |
| | | evidence of pericardial effusion |
| 7. Renal disorder | 1. | Persistent proteinuria >0.5 grams per day or > than 3+ if |
| | | quantitation not performed |
| | | OR |
| | 2. | Cellular casts--may be red cell, hemoglobin, granular, tubular, |
| | | or mixed |
| 8. Neurologic | 1. | Seizures--in the absence of offending drugs or known metabolic |
| disorder | | derangements; e.g., uremia, ketoacidosis, or electrolyte |
| | | imbalance |
| | | OR |
| | 2. | Psychosis--in the absence of offending drugs or known |
| | | metabolic derangements, e.g., uremia, ketoacidosis, or |
| | | electrolyte imbalance |
| 9. Hematologic | 1. | Hemolytic anemia--with reticulocytosis |
| disorder | | OR |
| | 2. | Leukopenia--<4,000/mm³ on ≧2 occasions |
| | | OR |
| | 3. | Lyphopenia--<1,500/mm³ on ≧2 occasions |
| | | OR |
| | 4. | Thrombocytopenia--<100,000/mm³ in the absence of offending |
| | | drugs |
| 10. Immunologic | 1. | Anti-DNA: antibody to native DNA in abnormal titer |
| disorder | | OR |
| | 2. | Anti-Sm: presence of antibody to Sm nuclear antigen |
| | | OR |
| | 3. | Positive finding of antiphospholipid antibodies on: |

| | |
|---|---|
| 1. | an abnormal serum level of IgG or IgM anticardiolipin |
| | antibodies, |
| 2. | a positive test result for lupus anticoagulant using a |
| | standard method, or |
| 3. | a false-positive test result for at least 6 months |
| | confirmed by Treponema pallidum immobilization or |
| | fluorescent treponemal antibody absorption test |

| | |
|---|---|
| 11. Positive | An abnormal titer of antinuclear antibody by immunofluorescence or an |
| antinuclear antibody | equivalent assay at any point in time and in the absence of drugs |

In some embodiments, a diagnosis of ISN/RPS 2003 Class III or IV LN is made according to Table 1, wherein the subject has either active or active/chronic disease, and wherein the diagnosis is confirmed by biopsy up to 3 months prior to diagnosis, and wherein the LN is active at diagnosis. Subjects are permitted to have co-existing Class V LN according to the ISN/RPS 2003 classification system.

Proteinuria and methods for measuring proteinuria are known to those of skill in the art. As used herein, proteinuria is expressed as a urine protein:creatinine ratio (uPCR).

Renal function is often assessed by measuring the glomerular filtration rate (GFR), which describes the flow rate of filtered fluid through the kidney. GFR is often reported as an estimated GFR, or eGFR. See, e.g., Levey A S et al. (March 1999) Annals of Internal Medicine 130(6):461-70.

### Muscle Atrophy

Skeletal muscle atrophy is defined as a progressive decrease in muscle mass that leads to weakness and impaired function. It occurs as a result of conditions of muscle disuse (e.g., immobilization, denervation, muscle unloading), aging, starvation, and a number of chronic disease states (e.g., chronic obstructive pulmonary disease and cancer). Regardless of the inciting event, skeletal muscle atrophy is characterized by a decrease in protein content, fiber diameter, force production, and endurance. Progressive loss of skeletal muscle mass may cause major physiological alterations. Muscle atrophy results in impaired functional strength, reduced insulin sensitivity, a decline in basal metabolic rate, and a concomitant increase in body fat mass. For these reasons, prolonged muscle disuse forms a significant health concern in several populations, with the elderly population being of particular relevance. Although it has been well established that age, nutrition, and physical activity are important factors regulating the maintenance of muscle mass, less progress has been made at developing effective pharmacological strategies to attenuate or even prevent muscle loss during periods of muscle disuse.

The TWEAK-Fn14 axis is as an important regulator of skeletal muscle wasting. Adult skeletal muscles express minimal levels of Fn14; however, conditions that cause atrophy rapidly induce the expression of Fn14. In addition, activation of the Fn14 signaling pathway by transgenic or exogenous administration of TWEAK promotes protein degradation and muscle atrophy. Moreover, TWEAK knock-out mice are partially protected from denervation-induced atrophy. Thus, inhibition of the TWEAK-Fn14 axis is a therapeutic strategy for prevention and/or treatment of conditions or diseases associated with muscle atrophy.

### Determination of Patient Population for Treatment of Muscle Atrophy

Skeletal muscle can atrophy in response to disuse, which may be secondary to conditions of nerve or blood supply deprivation and/or dug exposure such as glucocorticoids. In certain embodiments, the human subject to be treated has or is suspected of having skeletal muscle atrophy resulting from muscle disuse. In some instances, the muscle disuse results from immobilization of a limb of the subject. In certain embodiments, the human subject to be treated has skeletal muscle atrophy resulting from malnutrition. In other embodiments, the human subject to be treated has skeletal muscle atrophy resulting from long-term corticosteroid therapy. In certain embodiments, the human subject to be treated has skeletal muscle atrophy resulting from burns. In certain embodiments, the human subject to be treated has skeletal muscle atrophy resulting from renal failure. In some embodiments, the human subject has, is suspected of having, or is at risk of developing neurogenic atrophy.

Skeletal muscle can also atrophy in conditions of genetic or degenerative disorders. These conditions or disorders can be inflammatory or noninflammatory in nature. Muscular dystrophy constitutes a large group of hereditary myopathies characterized by atrophy and loss of muscle fibers in the absence of nerve disease; one common form that is included in this group is Duchenne's muscular dystrophy. Congenital muscle disease may also occur in the context of glycogen storage diseases, such as acid maltase deficiency, which results in babies with weak muscles, poor athletes, enlarged hearts, and often early death from cardiac failure. Congenital disorders leading to muscle atrophy also include, but are not limited to, mitochondrial myopathies, lipid myopathies, central tubular myopathies, and rhabdomyolysis. Myopathic conditions also may develop in adults, one of the most commonly observed being alcoholic myopathy. Skeletal muscle wasting also may occur as a component of neuronal disease, including but not limited to, amyotrophic lateral sclerosis (ALS). In addition, skeletal muscle wasting, also known as cachexia, is an important pathological condition seen in most terminally ill cancer patients and often is directly responsible for patients' death. Diseases of skeletal muscle that occur in the context of inflammation or autoimmunity include polymyositis, inflammatory myopathies, and glucocorticoid induced atrophy. Accordingly, in some embodiments, the human subject who has, or is suspected of having, or is at risk of developing muscle atrophy, has a disease such as cancer, acquired immunodeficiency syndrome (AIDS), congestive heart failure, chronic obstructive pulmonary disease (COPD), renal failure, liver disease, cachexia, alcohol-associated myopathy, amyotrophic lateral sclerosis (ALS), dermatomyositis, polymyositis, Guillain-Barre syndrome, motor neuropathy, muscular dystrophy, glycogen storage disease, mitochondrial myopathy, lipid myopathy, central tubular myopathy, rhabdomyolysis, alcoholic myopathy, inflammatory myopathy, glucocorticoid-induced myopathy, osteoarthritis, rheumatoid arthritis, spinal cord injury, stroke, inclusion body myositis, myotonic dystrophy, sarcopenia, diaphragm atrophy (e.g., resulting from intensive care unit stay), or other muscle atrophy resulting from intensive care unit stay. In these subjects, muscle atrophy results from a co-morbidity of one or more of these diseases.

In further embodiments, the human subject to be treated by the methods described herein has a diagnosis of skeletal muscle atrophy, and a soluble TWEAK level that is increased relative to a healthy control. Measurement of soluble TWEAK levels is described herein, and also in

WO 2006/138219, at, for example, pages 4-6, and 36.

### Anti-TWEAK Antibodies

In some embodiments, the anti-TWEAK antibody or antigen-binding fragment thereof comprises the six Complementarity Determining Regions (CDRs) from the murine and/or human P2D10 antibody disclosed in International Publication Number WO 2006/130374 at, for example, pages 1-13 and 44-48. In one embodiment, the anti-TWEAK antibody comprises/consists of the three heavy chain variable domain CDRs and the three light chain variable domain CDRs from a P2D10 antibody. Accordingly, the anti-TWEAK antibody may include all three of the P2D10 heavy chain variable domain CDRs, which are as follows:

| (SEQ ID NO: 1) | |
|---|---|
| CDR1: | GFTFSRYAMS, |
| (SEQ ID NO: 2) | |
| CDR2: | EISSGGSYPYYPDTVTG, |
| and | |
| (SEQ ID NO: 3) | |
| CDR3: | VLYYDYDGDRIEVMDY, |

and all three of the P2D10 light chain variable domain CDRs, which are as follows:

| (SEQ ID NO: 8) | |
|---|---|
| CDR1: | RSSQSLVSSKGNTYLH, |
| (SEQ ID NO: 9) | |
| CDR2: | KVSNRFS, |
| and | |
| (SEQ ID NO: 10) | |
| CDR3: | SQSTHFPRT. |

As used herein, CDRs refer to CDRs as defined by Chothia's hypervariable loops.

In some embodiments, the anti-TWEAK antibody includes a heavy chain variable domain comprising each of SEQ ID NOs:1, 2, and 3.

In some embodiments, the anti-TWEAK antibody includes a light chain variable domain comprising each of SEQ ID NOs:8, 9, and 10.

In some embodiments, the anti-TWEAK antibody includes a P2D10 heavy chain variable domain comprising the following sequence:

(SEQ ID NO: 17)
EVQLVESGGGLVQPGGSLRLSCAASGFTFSRYAMSWVRQAPGKGLEWVAE
ISSGGSYPYYPDTVTGRFTISRDNAKNSLYLQMNSLRAEDTAVYYCARVL
YYDYDGDRIEVMDYWGQGTLVTVSS
(huP2D10 H1 heavy chain variable domain).

In some embodiments, the antibody includes a P2D10 light chain variable domain comprising the following sequence:

(SEQ ID NO: 18)
DVVMTQSPLSLPVTPGEPASISCRSSQSLVSSKGNTYLHWYLQKPGQSPQ
FLIYKVSNRFSGVPDRFSGSGSGTDFTLKISRVEAEDVGVYFCSQSTHFP
RTFGGGTKVEIK
(huP2D10 L1 light chain variable domain)

In further embodiments, the antibody includes a P2D10 light chain variable domain comprising the following sequence:

(SEQ ID NO: 19)
DVVMTQSPLSLPVTPGEPASISCRSSQSLVSSKGNTYLHWYLQKPGQSPQ
LLIYKVSNRFSGVPDRFSGSGSGTDFTLKISRVEAEDVGVYYCSQSTHFP
RTFGGGTKVEIK
(huP2D10 L2 light chain variable domain)

In some embodiments, the anti-TWEAK antibody has a heavy chain variable domain comprising SEQ ID NO:17 and light chain variable domain comprising SEQ ID NO:18. In some embodiments, the anti-TWEAK antibody has a heavy chain variable domain comprising SEQ ID NO:17 and light chain variable domain comprising SEQ ID NO:19.

In some embodiments, the anti-TWEAK antibody has a heavy chain variable domain consisting, or consisting essentially of, SEQ ID NO:17, and light chain variable domain consisting, or consisting essentially of, SEQ ID NO:18 or SEQ ID NO:19. In some embodiments, the anti-TWEAK antibody has a heavy chain variable domain consisting of SEQ ID NO:17 and light chain variable domain consisting of SEQ ID NO:18 or SEQ ID NO:19.

In some embodiments, the heavy chain variable domain of the anti-TWEAK antibody includes an amino acid sequence which is at least 80%, 85%, 90%, 95%, 97%, 98%, 99% or more identical to SEQ ID NO:17 or which differs at least at 1 to 5 residues, but at fewer than 40, 30, 20, or 10 residues, from SEQ ID NO:17.

In some embodiments, the light chain variable domain of the anti-TWEAK antibody includes an amino acid sequence which is at least 80%, 85%, 90%, 95%, 97%, 98%, 99% or more identical to SEQ ID NO:18 or which differs at least at 1 to 5 residues, but at fewer than 40, 30, 20, or 10 residues, from SEQ ID NO:18.

In some embodiments, the light chain variable domain of the anti-TWEAK antibody includes an amino acid sequence which is at least 80%, 85%, 90%, 95%, 97%, 98%, 99% or more identical to SEQ ID NO:19 or which differs at least at 1 or 5 residues, but at fewer than 40, 30, 20, or 10 residues, from SEQ ID NO:19

In certain embodiments, the anti-TWEAK antibody is an antibody of the IgG1 isotype.

In some embodiments, the anti-TWEAK antibody includes a full-length P2D10 heavy chain comprising the following sequence:

(SEQ ID NO: 20)
EVQLVESGGGLVQPGGSLRLSCAASGFTFSRYAMSWVRQAPGKGLEWVA
EISSGGSYPYYPDTVTGRFTISRDNAKNSLYLQMNSLRAEDTAVYYCAR
VLYYDYDGDRIEVMDYWGQGTLVTVSSASTKGPSVFPLAPSSKSTSGGT
AALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKT
ISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALH
NHYTQKSLSLSPG
(huP2D10 H1 IgG1 heavy chain)

In some embodiments, the anti-TWEAK antibody includes a full-length P2D10 light chain comprising the following sequence:

(SEQ ID NO: 21)
DVVMTQSPLSLPVTPGEPASISCRSSQSLVSSKGNTYLHWYLQKPGQSP
QFLIYKVSNRFSGVPDRFSGSGSGTDFTLKISRVEAEDVGVYFCSQSTH
FPRTFGGGTKVEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC
(huP2D10 L1 light chain)

In further embodiments, the anti-TWEAK antibody includes a full-length P2D10 light chain comprising the following sequence:

(SEQ ID NO: 22)
DVVMTQSPLSLPVTPGEPASISCRSSQSLVSSKGNTYLHWYLQKPGQSP
QLLIYKVSNRFSGVPDRFSGSGSGTDFTLKISRVEAEDVGVYYCSQSTH
FPRTFGGGTKVEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC
(huP2D10 L2 light chain)

In some embodiments, the anti-TWEAK antibody has a heavy chain comprising SEQ ID NO:20 and light chain comprising SEQ ID NO:21. In some embodiments, the anti-TWEAK antibody has a heavy chain comprising SEQ ID NO:20 and light chain comprising SEQ ID NO:22.

In some embodiments, the anti-TWEAK antibody has a heavy chain consisting of SEQ ID NO:20 and light chain consisting of SEQ ID NO:21. In some embodiments, the anti-TWEAK antibody has a heavy chain consisting of SEQ ID NO:20 and light chain consisting of SEQ ID NO:22.

In some embodiments, the heavy chain of the anti-TWEAK antibody includes an amino acid sequence which is at least 80%, 85%, 90%, 95%, 97%, 98%, 99% or more identical to SEQ ID NO:20 or which differs at least at 1 to 5 residues, but at fewer than 40, 30, 20, or 10 residues, from SEQ ID NO:20.

In some embodiments, the light chain of the anti-TWEAK antibody includes an amino acid sequence which is at least 80%, 85%, 90%, 95%, 97%, 98%, 99% or more identical to SEQ ID NO:21 or which differs at least at 1 to 5 residues, but at fewer than 40, 30, 20, or 10 residues, from SEQ ID NO:21.

In some embodiments, the light chain of the anti-TWEAK antibody includes an amino acid sequence which is at least 80%, 85%, 90%, 95%, 97%, 98%, 99% or more identical to SEQ ID NO:22 or which differs at least at 1 to 5 residues, but at fewer than 40, 30, 20, or 10 residues, from SEQ ID NO:22.

In some embodiments, the anti-TWEAK antibody comprises, in the heavy chain variable domain, at least one, two, or three of SEQ ID NOs:1, 2, and 3. In particular, the heavy chain variable domain sequence may comprise SEQ ID NO:3. Alternatively, the heavy chain variable domain may include two of the sequences, e.g., it includes SEQ ID NOs:1 and 3, or it includes SEQ ID NOs:2 and 3.

In some embodiments, the anti-TWEAK antibody comprises, in the light chain variable domain, at least one, two, or three of SEQ ID NOs:8, 9, and 10. For example, the light chain variable domain sequence may comprise SEQ ID NO:10. Alternatively, the light chain variable domain may include two of the sequences, e.g., it includes SEQ ID NOs 8 and 10, or it includes SEQ ID NOs:9 and 10.

In some embodiments, the anti-TWEAK antibody includes, in the heavy chain variable domain sequence, at least one, two, or three of the following sequences within a CDR:

| (SEQ ID NO: 4) | |
|---|---|
| (i) | G-(YF)-(NT)-F-(STDN)-(RY)-Y-A-(MIL)-(HS), |
| (SEQ ID NO: 5) | |
| (ii) | Y-Y-(PV)-D-(TS)-V-(TK)-G |
| and | |
| (SEQ ID NO: 6) | |
| (iii) | (VL)-(IL)-(YF)-(YF)-D-(YF)-D |
| or | |
| (SEQ ID NO: 7) | |
| | (DE)-(RK)-(ILVM)(EQD)-(V AL)-M-(DE), |

where amino acids in parentheses represent alternatives for the particular position.

In some embodiments, the anti-TWEAK antibody includes, in the light chain variable domain sequence, at least one, two, or three of the following sequences within a CDR region:

(SEQ ID NO: 11)

| | |
|---|---|
| (i) | (RK)-S-S-Q-S-(LI)-(KV)-S-S-(KR)-G-N-(TN)-Y-L- |
| | (EHDNQY), |
| or | |

(SEQ ID NO: 12)

(RK)-S-S-Q-S-(LI)-V-S-S-(KR)-G-N-(TN)-Y-L-H

(SEQ ID NO: 13)

| | |
|---|---|
| (ii) | (KE)-(LVI)-S-(NYS)-(RW)-(FAD)-S, |
| or | |

(SEQ ID NO: 14)

| | |
|---|---|
| | K(LVI)-S-(NYS)-R-(FAD)-S, |
| and | |

(SEQ ID NO: 15)

| | |
|---|---|
| (iii) | (SM)-Q-(GSA)-(ST)-(HEQ)-(FWL)-P |
| or | |

(SEQ ID NO: 16)

S-Q-(GSA)-(SIT)-(HEQ)-F-P,

where amino acids in parentheses represent alternatives for the particular position.

In some embodiments, the anti-TWEAK antibody includes a heavy chain variable domain comprising the sequence of each of SEQ ID NOs:1, 2, and 3, wherein each sequence contains from zero to four modifications (e.g., substitutions, insertions or deletions) per CDR. In further embodiments, the anti-TWEAK antibody includes a light chain variable domain comprising the sequence of each of SEQ ID NOs:8, 9, and 10, wherein each sequence contains from zero to four modifications (e.g., substitutions, insertions or deletions) per CDR.

The antibody can be a human, humanized, CDR-grafted, chimeric, mutated, affinity matured, deimmunized, synthetic or otherwise in vitro-generated antibody, and combinations thereof. In some embodiments, the anti-TWEAK antibody is a humanized antibody. For example, the anti-TWEAK antibody may be a CDR-grafted antibody comprising the CDRs of the mouse P2D10 antibody described in International Publication Number WO 2006/130374 (i.e., SEQ ID NOs:1, 2, and 3, and SEQ ID NOs:8, 9, and 10 for light chain CDRs), or variants thereof, grafted into human heavy and light chain variable domains to create an antibody with mouse P2D10 CDRs and human framework regions in the variable domain.

In one embodiment, the heavy chain framework (e.g., FR1, FR2, FR3, individually, or a sequence encompassing FR1, FR2, and FR3, but excluding CDRs) includes an amino acid sequence which is at least 80%, 85%, 90%, 95%, 97%, 98%, 99% or more identical to the heavy chain framework of one of the following germline V segment sequences: DP-25, DP-1, DP-12, DP-9, DP-7, DP-31, DP-32, DP-33, DP-58, DP-54, other VH I subgroup germline sequence, other VH III subgroup germline sequence, or another V gene which is compatible with the canonical structure class 1-3 (see, e.g., Chothia et al. (1992) J. Mol. Biol. 227:799-817; Tomlinson et al. (1992) J. Mal Biol. 227:776-798). Other frameworks compatible with the canonical structure class 1-3 include frameworks with the one or more of the following residues according to Kabat numbering: Ala, Gly, Thr, or Val at position 26; Gly at position 26; Tyr, Phe, or Gly at position 27; Phe, Val, Ile, or Leu at position 29; Met, Ile, Leu, Val, Thr, Trp, or Ile at position 34; Arg, Thr, Ala, Lys at position 94; Gly, Ser, Asn, or Asp at position 54; and Arg at position 71.

In some embodiments, the light chain framework (e.g., FR1, FR2, FR3, individually, or a sequence encompassing FR1, FR2, and FR3, but excluding CDRs) includes an amino acid sequence, which is at least 80%, 85%, 90%, 95%, 97%, 98%, 99% or more identical to the light chain framework of a VκII subgroup germline sequence or one of the following germline V segment sequences: A17, A1, A18, A2, A19/A3, A23, a VκI subgroup germline sequence (e.g., a DPK9 sequence), or another V gene which is compatible with the canonical structure class 4-1 (see, e.g., Tomlinson et al. (1995) EMBO J. 14:4628). Other frameworks compatible with the canonical structure class 4-1 include frameworks with the one or more of the following residues according to Kabat numbering: Val or Leu or Ile at position 2; Ser or Pro at position 25; Ile or Leu at position 27b; Gly at position 29; Phe or Leu at position 33; and Phe at position 71. Further, according to the Kabat numbering, position 48 can be Ile or Val.

In another embodiment, the light chain framework (e.g., FR1, FR2, FR3, individually, or a sequence encompassing FR1, FR2, and FR3, but excluding CDRs) includes an amino acid sequence, which is at least 80%, 85%, 90%, 95%, 97%, 98%, 99% or more identical to the light chain framework of a VκI subgroup germline sequence, e.g., a DPK9 sequence.

In some embodiments, the light or the heavy chain variable framework (e.g., the region encompassing at least FR1, FR2, FR3, and optionally FR4) can be chosen from: (a) a light or heavy chain variable framework including at least 80%, 90%, 95%, or preferably 100% of the amino acid residues from a human light or heavy chain variable framework, e.g., a light or heavy chain variable framework residue from a human mature antibody, a human germline sequence, a human consensus sequence, or a human antibody described herein; (b) a light or heavy chain variable framework including from 20% to 80%, 40% to 60%, 60% to 90%, or 70% to 95% of the amino acid residues from a human light or heavy chain variable framework, e.g., a light or heavy chain variable framework residue from a human mature antibody, a human germline sequence, a human consensus sequence; (c) a non-human framework (e.g., a rodent framework); or (d) a non-human framework that has been modified, e.g., to remove antigenic or cytotoxic determinants, e.g., deimmunized, or partially humanized. In one embodiment, the heavy chain variable domain sequence includes human residues or human consensus sequence residues at one or more of the following positions (preferably at least five, ten, twelve, or all): (in the FR of the variable domain of the light chain) 4L, 35L, 36L, 38L, 43L, 44L, 58L, 46L, 62L, 63L, 64L, 65L, 66L, 67L, 68L, 69L, 70L, 71L, 73L, 85L, 87L, 98L, and/or (in the FR of the variable domain of the heavy chain) 2H, 4H, 24H, 36H, 37H, 39H, 43H, 45H, 49H, 58H, 60H, 67H, 68H, 69H, 70H, 73H, 74H, 75H, 78H, 91H, 92H, 93H, and/or 103H (according to the Kabat numbering).

In some embodiments, the anti-TWEAK antibody includes at least one non-human CDR, e.g., a murine CDR, e.g., a CDR from the P2D10 antibody as described in International Publication Number WO 2006/130374, or a variant thereof, and at least one framework which differs from a framework of P2D10 by at least one amino acid, e.g., at least 5, 8, 10, 12, 15, or 18 amino acids. For example, the anti-TWEAK antibody may include one, two, three, four, five, or six such non-human CDRs and include at least one amino acid difference in at least three of HC FR1, HC FR2, HC FR3, LC FR1, LC FR2, and LC FR3.

In some embodiments, one or both of the variable domains include amino acid positions in the framework region that are variously derived from both a murine antibody (e.g., P2D10) and a humanized antibody (e.g., 56-84m and K107) or germline sequence. For example, the variable domain will include a number of positions at which the amino acid is identical to both the murine P2D10 antibody and the human antibody (or germline sequence) because the two are identical at that position. Of the remaining framework positions where murine P2D10 and the human antibody differ, at least 50, 60, 70, 80, or 90% of the positions of the variable domain are preferably identical to the human antibody (or germline sequence) rather than the murine. None, or at least one, two, three, or four of such remaining framework positions may be identical to the murine P2D10 antibody rather than to the human antibody. For example, in HC FR1, one or two such positions can be murine; in HC FR2, one or two such positions can be murine; in FR3, one, two, three, or four such positions can be murine; in LC FR1, one, two, three, or four such positions can be murine; in LC FR2, one or two such positions can be murine; in LC FR3, one or two such positions can be murine. The heavy and light chains of the anti-TWEAK antibody can be full-length or substantially full-length. The protein can include at least one, and preferably two, complete heavy chains, and at least one, and preferably two, complete light chains. The antibody can include an antigen-binding fragment (e.g., a Fab, F(ab′)₂, Fv or a single chain Fv fragment. In yet other embodiments, the antibody has a heavy chain constant region chosen from, e.g., IgG1, IgG2, IgG3, IgG4, IgM, IgA1, IgA2, IgD, and IgE; particularly, chosen from, e.g., IgG1, IgG2, IgG3, and IgG4, more particularly, IgG1 (e.g., human IgG1). Typically, the heavy chain constant region is human or a modified form of a human constant region. In further embodiments, the antibody has a light chain constant region chosen from, e.g., a kappa or lambda light chain, particularly kappa light chain (e.g., human kappa). In one embodiment, the constant region of an anti-TWEAK antibody may be modified by mutation of one or more amino acid residues to impart a desired functional property (e.g., altered effector function or half-life) using methods well known in the art.

### Non-TWEAK Related Agents

The non-TWEAK-related agents described herein for use in treatment of lupus nephritis may be a steroid, including corticosteroids and gluco-corticosteroids. The steroid may be selected from prednisone, prednisolone, methylprednisolone, hydrocortisone, and dexamethasone. Other steroids are well known and encompassed in the methods and compositions described herein. In one embodiment, the steroid is provided orally or intravenously. In some embodiments, the steroid is provided at a low dose, e.g., less than 10 mg/day, e.g., less than 7.5 mg per day. In other embodiments, the steroid is provided at a medium dose, e.g., between 7.5 and 15 mg per day. In still other embodiments, the steroid is provided at a high dose, e.g., greater than 15 mg per day.

The non-TWEAK-related agent for use in treatment of lupus nephritis may also be an immunosuppressant. The immunosuppressant may be selected from mycophenolate mofetil (MMF), cyclophosphamide, azathioprine, and cyclosporine. Other immunosuppressants are well known and are encompassed in the methods described herein. In some embodiments, the immunosuppressant is provided at a low, intermediate, or high dose. Low, intermediate, and high doses will vary between immunosuppressants, and will be known to the skilled artisan. In one exemplary embodiment, the immunosuppressant is MMF. In one embodiment, a low dose of MMF is less than or equal to 500 mg, an intermediate dose is greater than 500 mg and less than or equal to 3 grams, and a high dose is greater than 3 grams.

The non-TWEAK-related agent for use in treatment of muscle atrophy can be a branched amino acid (e.g., leucine, isoleucine, valine, or lysine).

Another non-TWEAK-related agent for use in treatment of muscle atrophy can be a selective androgen receptor modulator (SARM). Non-limiting examples of SARMs include tamoxifen, enobosarm, BMS-564,929, LGD-4033, AC-262,356, JNJ-28330835, LGD-2226, LGD-3303, S-40503, and S-23.

Another non-TWEAK-related agent for use in treatment of muscle atrophy can be a low molecular weight heparin (LMWH). Such LMWH's are useful in thromoboprophylaxis to reduce the risk of deep vein thrombosis or pulmonary embolism. Enoxaparin is an exemplary LMWH.

Another non-TWEAK-related agent for use in treatment of muscle atrophy can be an agent that induces hypertrophy, such as a myostatin pathway inhibitor.

### Combination Therapy

In one embodiment, the anti-TWEAK antibody may be administered in combination with one or more non-TWEAK-related agents. In this embodiment, the anti-TWEAK antibody and one or more additional agents are administered to a subject at the same time or within a certain interval of one another, such that there is overlap of an effect of each agent on the patient. Preferably, the administrations of the anti-TWEAK antibody and the additional agent or agents are spaced sufficiently close together such that a combinatorial effect is achieved. The interval can be an interval of hours, days, or weeks. Generally, the agents are concurrently bioavailable in the subject, i.e., the agents may each be detected in the subject at the same time. In one embodiment, at least one administration of one of the agents, e.g., the anti-TWEAK antibody, is made while a second agent is still present at a therapeutic level in the subject.

In some embodiments, the anti-TWEAK antibody is administered between an earlier and a later administration of an additional agent. In other embodiments, an additional agent is administered between an earlier and a later administration of the anti-TWEAK antibody. In a preferred embodiment, at least one administration of one of the agents, e.g., the anti-TWEAK antibody is made within 1, 7, 14, 30, or 60 days of the additional agent.

In some embodiments, prior to administering the anti-TWEAK antibody and one or more additional agents, the subject was receiving a non-TWEAK related agent. The subject may have had a response that did not meet a predetermined threshold. In other embodiments, the subject can be one who has not been previously administered either an anti-TWEAK antibody or a non-TWEAK-related agent prior to being administered the anti-TWEAK antibody and a non-TWEAK-related agent in combination.

In one implementation, the anti-TWEAK antibody and one or more non-TWEAK related agents are provided as a co-formulation, and the co-formulation is administered to the subject. It is further possible, e.g., at least 24 hours before or after administering the co-formulation, to administer one of the agents separately from the other. In another implementation, the anti-TWEAK antibody and one or more non-TWEAK related agents are provided as separate formulations, and the step of administering includes sequentially administering the agents. The sequential administrations can be provided on the same day (e.g., within one hour of one another or at least 3, 6, or 12 hours apart) or on different days.

The anti-TWEAK antibody and the one or more non-TWEAK related agents may each be administered as a plurality of doses separated in time, e.g., according to a regimen. The regimen for one or both may have a regular periodicity. The regimen for an additional agent can have a different periodicity from the regimen for the anti-TWEAK antibody, e.g., one can be administered more frequently than the other. The agents can be administered by any appropriate method, e.g., subcutaneously, intramuscularly, or intravenously. The subject can be administered doses of an additional agent and doses of the anti-TWEAK antibody for greater than 14 weeks, greater than six or nine months, greater than 1, 1.5, or 2 years.

In some embodiments, the anti-TWEAK antibody and one or more non-TWEAK related agent is administered at about the same dose as the dose used for monotherapy. In other embodiments, the non-TWEAK related agent is administered at a dosage that is equal to or less than an amount required for efficacy if administered alone (e.g., at least 10, 20, 30, or 40% less). For example, in some embodiments in which the subject has previously received a non-TWEAK-related agent, the subject is administered a reduced dose of that non-TWEAK related therapy after receiving the anti-TWEAK antibody (relative to the dose of the non-TWEAK related therapy received before receiving the anti-TWEAK antibody for the first time).

A subject can be evaluated after receiving the first and second agent, e.g., for indicia of responsiveness. A skilled artisan can use various clinical or other indicia of effectiveness of treatment. The subject can be monitored at various times during a regimen.

An anti-TWEAK antibody as described herein and a non-TWEAK-related agent may be administered in the same or separate pharmaceutical compositions which comprise a “therapeutically effective amount” of an anti-TWEAK antibody and/or a “therapeutically effective amount” of one or more non-TWEAK related agents. In one embodiment, the therapeutically effective amount of the anti-TWEAK antibody for treatment of lupus nephritis or muscle atrophy is 20 mg/kg. In another embodiment, the therapeutically effective amount of the anti-TWEAK antibody for treatment of lupus nephritis is 3 mg/kg.

### Successful Treatment of Lupus Nephritis

In one embodiment, a subject is said to be successfully treated (i.e., to have received a “therapeutically effective amount” of an agent or combination of agents) when there is a complete renal response, characterized by urinary protein:creatine ratio (uPCR) less than 0.5 with greater than or equal to 50% reduction of uPCR from baseline (i.e., the uPCR of the subject prior to treatment with an anti-TWEAK antibody or the uPCR of a healthy control) and estimated eGFR within normal range.

In other embodiments, a subject is said to be successfully treated when there is a partial renal response characterized by a greater than or equal to 50% reduction in uPCR from baseline with one of the following: a) uPCR of less than 1.0 if the day 1 (baseline) was greater than or equal to 3.0, or b) uPCR greater than 3.0 if the Day 1 (baseline) ratio was greater than 3.0; and stabilization of renal function (eGFR plus or minus 25% of day 1 (baseline) or serum creatinine within normal range.

### Successful Treatment of Muscle Atrophy

The following are exemplary methods that can be used to determine if a human subject has been successfully treated with an anti-TWEAK antibody therapy (either alone or in combination with other agent(s)).

In one embodiment, a subject is said to be successfully treated when there is a change in the magnitude of muscle atrophy at least one, or at least two, months after start of treatment. The percentage change in the magnitude of muscle atrophy can be determined by, e.g., T1-weighted magnetic resonance imaging (T1W-MRI) analysis of the cross-sectional area of the muscle under examination.

In another embodiment, a subject is said to be successfully treated when there is an improvement in isometric knee-extension strength and/or isometric plantar-flexion strength as measured by e.g., dynamometry.

In one embodiment, a subject is said to be successfully treated when there is a change in total cross-sectional area of type I and type II muscle fibers as measured by histological analysis of muscle biopsy.

In yet another embodiment, a subject is said to be successfully treated when there is a change in recovery of muscle oxidative metabolism as measured by, e.g., near-infrared spectroscopy of the muscle being treated.

In a certain embodiment, a subject is said to be successfully treated when there is a change in biomarkers related to muscle atrophy compared to baseline or different time points during treatment. Such biomarkers include, but are not limited to, follistatin, myostatin, and C-terminal agrin fragment (CAF).

In a further embodiment, a subject is said to be successfully treated when the subject is determined by a health care practitioner to improve compared with baseline (day of or the day before treatment commences) in timed functional activity performance such as by the five times sit-to-stand test (FTSST), the timed-up-and-go (TUG) test, and the stair climbing test (SCT).

### Evaluating a Subject for Tweak or Fn14

Techniques for evaluating a subject for TWEAK in a biological sample of the subject are described in WO 2006/138219. Such techniques can include detecting the presence, levels, expression or activity of a TWEAK, e.g., by qualitative or quantitative analysis of mRNA, cDNA, or protein, or by evaluating one or more nucleotides in a nucleic acid (genomic, mRNA, or cDNA) encoding TWEAK or TWEAK-R. Such techniques include methods for protein detection (e.g., Western blot or ELISA), and hybridization-based methods for nucleic acid detection (e.g., PCR or Northern blot). For example, an immunoassay can be used to detect TWEAK protein, e.g., in a urine sample of the subject. In other embodiments, the method can include administering a labeled TWEAK or TWEAK-R binding agent (e.g., an antibody) to a subject, and evaluating localization of the labeled binding agent in the subject, e.g., by imaging the subject (e.g., imaging at least a portion of the kidney of the subject). The expression level of a TWEAK can be determined using an antibody specific for TWEAK (e.g., using a western blot or an ELISA assay).

### Pharmaceutical Compositions

An anti-TWEAK antibody can be formulated as a pharmaceutical composition, e.g., for administration to a subject to treat lupus nephritis. Typically, a pharmaceutical composition includes a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. The composition can include a pharmaceutically acceptable salt, e.g., an acid addition salt or a base addition salt (see e.g., Berge, S. M., et al. (1977) J. Pharm. Sci. 66:1-19).

The anti-TWEAK antibody can be formulated according to standard methods. Pharmaceutical formulation is a well-established art, and is further described, e.g., in Gennaro (ed.), Remington: The Science and Practice of Pharmacy, 20th ed., Lippincott, Williams & Wilkins (2000) (ISBN: 0683306472); Ansel et al., Pharmaceutical Dosage Forms and Drug Delivery Systems, 7th Ed., Lippincott Williams & Wilkins Publishers (1999) (ISBN: 0683305727); and Kibbe (ed.), Handbook of Pharmaceutical Excipients American Pharmaceutical Association, 3rd ed. (2000) (ISBN: 091733096X).

In one embodiment, the anti-TWEAK antibody can be formulated with excipient materials, such as sodium chloride, sodium dibasic phosphate heptahydrate, sodium monobasic phosphate, and a stabilizer. It can be provided, for example, in a buffered solution at a suitable concentration and can be stored at 2-8° C.

The pharmaceutical compositions may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form can depend on the intended mode of administration and therapeutic application. Typically compositions for the agents described herein are in the form of injectable or infusible solutions.

In a certain embodiment, the anti-TWEAK antibody is supplied as a sterile liquid drug product at a concentration of 100 mg/mL in sodium succinate (pH 5.5), succinic acid, L-arginine, and polysorbate 80. In some embodiments, the anti-TWEAK antibody is provided in 3 mL vials containing 1 mL of 100 mg/mL anti-TWEAK antibody. In some embodiments, the anti-TWEAK antibody is provided in composition containing a fixed dose of 1,600 mg or 240 mg of an anti-TWEAK antibody and a pharmaceutically acceptable carrier.

Such compositions can be administered by a parenteral mode (e.g., intravenous, subcutaneous, intraperitoneal, or intramuscular injection). The phrases “parenteral administration” and “administered parenterally” as used herein mean modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

The composition can be formulated as a solution, microemulsion, dispersion, liposome, or other ordered structure suitable for stable storage at high concentration. Sterile injectable solutions can be prepared by incorporating an agent described herein in the required amount in an appropriate solvent with one or a combination of ingredients enumerated above, as required, followed by filtered sterilization. Generally, dispersions are prepared by incorporating an agent described herein into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of an agent described herein plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In certain embodiments, the anti-TWEAK antibody may be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York, 1978.

An anti-TWEAK antibody can be modified, e.g., with a moiety that improves its stabilization and/or retention in circulation, e.g., in blood, serum, or other tissues, e.g., by at least 1.5, 2, 5, 10, or 50 fold. The modified blocking agent can be evaluated to assess whether it can reach sites of damage after a stroke (e.g., by using a labeled form of the blocking agent).

For example, the anti-TWEAK antibody can be associated with a polymer, e.g., a substantially non-antigenic polymer, such as a polyalkylene oxide or a polyethylene oxide. Suitable polymers will vary substantially by weight. Polymers having molecular number average weights ranging from about 200 to about 35,000 Daltons (or about 1,000 to about 15,000, and 2,000 to about 12,500) can be used.

For example, an anti-TWEAK antibody can be conjugated to a water-soluble polymer, e.g., a hydrophilic polyvinyl polymer, e.g. polyvinylalcohol or polyvinylpyrrolidone. A non-limiting list of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene (Pluronics); polymethacrylates; carbomers; and branched or unbranched polysaccharides.

When the anti-TWEAK antibody is used in combination with a second agent, the two agents can be formulated separately or together. For example, the respective pharmaceutical compositions can be mixed, e.g., just prior to administration, and administered together or can be administered separately, e.g., at the same or different times.

### Administration

The anti-TWEAK antibody can be administered to a subject, e.g., a human subject, by a variety of methods. For many applications, the route of administration is one of: intravenous injection or infusion (IV), subcutaneous injection (SC), intraperitoneally (IP), or intramuscular injection. In some cases, administration may be directly into the CNS, e.g., intrathecal or intracerebroventricular (ICV). The anti-TWEAK agent can be administered as a fixed dose (i.e., independent of the weight of the patient), or in a mg/kg dose (i.e., a dose which varies based on the weight of the subject).

In one embodiment, for treating lupus nephritis or muscle atrophy, the dosage of the anti-TWEAK antibody is 20 mg/kg. In another embodiment, for treating lupus nephritis, the dosage of the anti-TWEAK antibody is 3 mg/kg. Fixed doses corresponding to these concentrations may also be prepared.

The route and/or mode of administration of the anti-TWEAK antibody can also be tailored for the individual case, e.g., by monitoring the subject, e.g., using assessment criteria discussed herein.

The dose may be administered every 2 months, every 6 weeks, monthly, biweekly, weekly, or daily, as appropriate, over a period of time to encompass at least 2 doses, 3 doses, 5 doses, 10 doses, or more.

Dosage unit form or “fixed dose” as used herein refers to physically discrete units suited as unitary dosages for the subjects to be treated; each unit contains a predetermined quantity of active compound calculated to produce the desired therapeutic effect in association with the required pharmaceutical carrier and optionally in association with the other agent.

Single or multiple dosages may be given. Alternatively, or in addition, the blocking agent may be administered via continuous infusion. The treatment can continue for days, weeks, months or even years.

A pharmaceutical composition may include a “therapeutically effective amount” of an agent described herein. Such effective amounts can be determined based on the effect of the administered agent, or the combinatorial effect of agents if more than one agent is used. A therapeutically effective amount of an agent may also vary according to factors such as the disease state, age, sex, and weight of the individual, and the ability of the compound to elicit a desired response in the individual. A therapeutically effective amount is also one in which any toxic, or detrimental effects, of the composition is outweighed by the therapeutically beneficial effects. In one embodiment, the therapeutically effective amount of the anti-TWEAK antibody is 20 mg/kg. In another embodiment, the therapeutically effective amount of the anti-TWEAK antibody is 3 mg/kg.

In one exemplary administration regime, subjects having or suspected of having lupus nephritis that are currently being administered a steroid and an immunosuppressant according to the standard of care are administered 20 mg/kg or 3 mg/kg of the anti-TWEAK antibody described herein. The anti-TWEAK antibody is administered parenterally every four weeks for at least 48 weeks. In one embodiment, the steroid (e.g. prednisone) is administered at a low dose, e.g., less than 7.5 mg/day. In one embodiment, this represents a reduction in the amount of steroid that the patient previously received. In one embodiment, the immunosuppressant (e.g., MMF) is also administered at a low dose, e.g., less than or equal to 500 grams. In one embodiment, this represents a reduction in the amount of immunosuppressant that the patient previously received. In one embodiment, the subject that is currently being administered prednisone and an immunosuppressant (e.g., MMF) continues to receive the steroid and an immunosuppressant (e.g., MMF) throughout treatment with the anti-TWEAK antibody, so that the anti-TWEAK antibody is considered to be an “add-on” treatment to background therapy.

In another exemplary administration regime, subjects having or suspected of having muscle atrophy are administered 20 mg/kg of the anti-TWEAK antibody described herein. The anti-TWEAK antibody is administered intravenously in 4 single doses of 20 mg/kg at two, three or four week intervals. In certain instances, where the subject's limbs are immobilized (e.g., in a brace), the subject is also administered enoxaparin (40 mg QD) by subcutaneous injection during the immobilization period.

### Devices and Kits

Pharmaceutical compositions that comprise the anti-TWEAK antibody alone or in combination with non-TWEAK related agent(s) can be administered with a medical device. The device can be designed with features such as portability, room temperature storage, and ease of use so that it can be used in emergency situations, e.g., by an untrained subject or by emergency personnel in the field, removed to medical facilities and other medical equipment. The device can include, e.g., one or more housings for storing pharmaceutical preparations that include anti-TWEAK antibody, and can be configured to deliver one or more unit doses of the blocking agent.

For example, the pharmaceutical composition can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other devices, implants, delivery systems, and modules are also known.

An anti-TWEAK antibody alone or in combination with non-TWEAK related agents can be provided in a kit. In one embodiment, the kit includes (a) a container that contains a composition that includes an anti-TWEAK antibody alone or in combination with one or more non-TWEAK related agents, and optionally (b) informational material. The informational material can be descriptive, instructional, marketing or other material that relates to the methods described herein and/or the use of the agents for therapeutic benefit. The kit may also comprise a first container that contains a composition that includes the anti-TWEAK antibody, and a second container that includes a non-TWEAK-related agent or agents.

In addition to the anti-TWEAK antibody, the composition in the kit can include other ingredients, such as a solvent or buffer, a stabilizer, or a preservative. The blocking agent can be provided in any form, e.g., liquid, dried or lyophilized form, preferably substantially pure and/or sterile. When the agents are provided in a liquid solution, the liquid solution preferably is an aqueous solution. When the agents are provided as a dried form, reconstitution generally is by the addition of a suitable solvent. The solvent, e.g., sterile water or buffer, can optionally be provided in the kit.

The kit can include one or more containers for the composition or compositions containing the agents. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the agents. The containers can include a combination unit dosage, e.g., a unit that includes both the anti-TWEAK antibody and the second agent, e.g., in a desired ratio. For example, the kit includes a plurality of syringes, ampules, foil packets, blister packs, or medical devices, e.g., each containing a single combination unit dose. The containers of the kits can be air tight, waterproof (e.g., impermeable to changes in moisture or evaporation), and/or light-tight.

The kit optionally includes a device suitable for administration of the composition, e.g., a syringe or other suitable delivery device. The device can be provided pre-loaded with one or both of the agents or can be empty, but suitable for loading.

A commercial package can be prepared comprising a combination described herein together with instructions for simultaneous, separate or sequential use.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating lupus nephritis comprising administering 20 mg/kg or 3 mg/kg of an anti-TWEAK antibody to a human subject having or suspected of having lupus nephritis, thereby treating lupus nephritis.

2. The method of claim 1, wherein the subject receiving the anti-TWEAK antibody is currently being administered a non-TWEAK related agent.

3. The method of claim 2, wherein the non-TWEAK-related agent is a steroid or an immunosuppressant.

4-5. (canceled)

6. The method of claim 1, wherein the anti-TWEAK antibody comprises complementarity determining regions (CDRs) selected from the group consisting of: (i) the CDRs shown in SEQ ID NOs:1, 2, 3, 8, 9, and 10; and (ii) the CDRs shown in SEQ ID NOs:4, 5, 6 or 7, 11 or 12, 13 or 14, and 15 or 16.

7-16. (canceled)

17. The method of claim 1, wherein the human subject having or suspected of having lupus nephritis has been diagnosed with systemic lupus erythematosus (SLE), and has been diagnosed with Class III or Class IV LN according to the ISN/RPS 2003, and has a proteinuria uPCR greater than 1.0.

18. The method of claim 17, wherein the SLE is diagnosed by meeting at least 4 of the criteria documented by the American College of Rheumatology (ACR) as criteria for SLE, as outlined in Table 2, wherein one of the criteria must be a positive antinuclear antibody (ANA), anti-Sm, or anti-dsDNA antibody.

19. A composition comprising:
a fixed dose of 1,600 mg or 240 mg of an anti-TWEAK antibody together with pharmaceutically acceptable carrier;
a fixed dose of 1,600 mg or 240 mg of an anti-TWEAK antibody and a steroid together with a pharmaceutically acceptable carrier;
a fixed dose of 1,600 mg or 240 mg of an anti-TWEAK antibody and an immunosuppressant together with a pharmaceutically acceptable carrier; or
a fixed dose of 1,600 mg or 240 mg of an anti-TWEAK antibody and a steroid and an immunosuppressant together with a pharmaceutically acceptable carrier.

20-28. (canceled)

29. A kit comprising the composition of claim 19, together with instructions for use in treating lupus nephritis.

30. (canceled)

31. A method for treating muscle atrophy in a human subject in need thereof, the method comprising administering 20 mg/kg of an anti-TWEAK antibody to the human subject.

32. The method of claim 31, wherein the human subject is also administered an amino acid therapy.

33. The method of claim 32, wherein the amino acid therapy comprises administration of an amino acid selected from the group consisting of leucine, isoleucine, valine, and lysine.

34. The method of claim 31, wherein the human subject is also administered a selective androgen receptor modulator (SARM).

35. The method of claim 34, wherein the SARM is selected from the group consisting of: tamoxifen, enobosarm, BMS-564,929, LGD-4033, AC-262,356, JNJ-28330835, LGD-2226, LGD-3303, S-40503, and S-23.

36. The method of claim 31, wherein the human subject is also administered a low molecular weight heparin or a myostatin pathway inhibitor.

37. The method of claim 31, wherein the human subject has a disease selected from the group consisting of cancer, acquired immunodeficiency syndrome (AIDS), congestive heart failure, chronic obstructive pulmonary disease (COPD), renal failure, liver disease, cachexia, alcohol-associated myopathy, amyotrophic lateral sclerosis (ALS), dermatomyositis, polymyositis, Guillain-Barre syndrome, motor neuropathy, muscular dystrophy, glycogen storage disease, mitochondrial myopathy, lipid myopathy, central tubular myopathy, rhabdomyolysis, alcoholic myopathy, inflammatory myopathy, glucocorticoid-induced myopathy, osteoarthritis, rheumatoid arthritis, spinal cord injury, stroke, inclusion body myositis, myotonic dystrophy, sarcopenia, or diaphragm atrophy.

38. The method of claim 31, wherein the human subject has, is suspected of having, or is at risk of developing disuse muscle atrophy.

39. The method of claim 31, wherein the human subject has, is suspected of having, or is at risk of developing neurogenic atrophy.

40. The method of claim 31, wherein the human subject is immobilized.

41. The method of claim 31, wherein the human subject has malnutrition.

42. The method of claim 31, wherein the human subject has undergone long-term corticosteroid therapy.

43. The method of claim 31, wherein the human subject has suffered a burn.

44-46. (canceled)

47. The method of claim 31, wherein the anti-TWEAK antibody comprises:
(i) a heavy chain variable domain (VH) complementarity determining region (CDR) 1 comprising the amino acid sequence set forth in SEQ ID NO:4, a VH CDR2 comprising the amino acid sequence set forth in SEQ ID NO:5, and a VH CDR3 comprising the amino acid sequence set forth in SEQ ID NO:6 or 7; and
(ii) a light chain variable domain (VL) CDR1 comprising the amino acid sequence set forth in SEQ ID NO:11 or 12, a VL CDR2 comprising the amino acid sequence set forth in SEQ ID NO:13 or 14, and a VL CDR3 comprising the amino acid sequence set forth in SEQ ID NO:15 or 16.

48. The method of claim 31, wherein the anti-TWEAK antibody comprises:
(i) a VH CDR1 comprising the amino acid sequence set forth in SEQ ID NO:1, a VH CDR2 comprising the amino acid sequence set forth in SEQ ID NO:2, and a VH CDR3 comprising the amino acid sequence set forth in SEQ ID NO:3; and
(ii) a VL CDR1 comprising the amino acid sequence set forth in SEQ ID NO:8, a VL CDR2 comprising the amino acid sequence set forth in SEQ ID NO:9, and a VL CDR3 comprising the amino acid sequence set forth in SEQ ID NO:10.

49-50. (canceled)

51. A composition comprising:
a fixed dose of 1,600 mg of an anti-TWEAK antibody and a selective androgen receptor modulator;
a fixed dose of 1,600 mg of an anti-TWEAK antibody and a branched amino acid;
a fixed dose of 1,600 mg of an anti-TWEAK antibody and a low molecular weight heparin or a myostatin pathway inhibitor.

52-56. (canceled)


SEQUENCE LISTING (22 sequences)

SEQ ID NO: 1 - protein, 10 aa, Artificial Sequence
  source: /note=“Description of Artificial Sequence Synthetic peptide”
GFTFSRYAMS

SEQ ID NO: 2 - protein, 17 aa, Artificial Sequence
  source: /note=“Description of Artificial Sequence Synthetic peptide”
EISSGGSYPYYPDTVTG

SEQ ID NO: 3 - protein, 16 aa, Artificial Sequence
  source: /note=“Description of Artificial Sequence Synthetic peptide”
VLYYDYDGDRIEVMDY

SEQ ID NO: 4 - protein, 10 aa, Artificial Sequence
  source: /note=“Description of Artificial Sequence Synthetic peptide”
GYNFSRYAMH

SEQ ID NO: 5 - protein, 8 aa, Artificial Sequence
  source: /note=“Description of Artificial Sequence Synthetic peptide”
YYPDTVTG

SEQ ID NO: 6 - protein, 7 aa, Artificial Sequence
  source: /note=“Description of Artificial Sequence Synthetic peptide”
VIYYDYD

SEQ ID NO: 7 - protein, 7 aa, Artificial Sequence
  source: /note=“Description of Artificial Sequence Synthetic peptide”
DRIEVMD

SEQ ID NO: 8 - protein, 16 aa, Artificial Sequence
  source: /note=“Description of Artificial Sequence Synthetic peptide”
RSSQSLVSSKGNTYLH

SEQ ID NO: 9 - protein, 7 aa, Artificial Sequence
  source: /note=“Description of Artificial Sequence Synthetic peptide”
KVSNRFS

SEQ ID NO: 10 - protein, 9 aa, Artificial Sequence
  source: /note=“Description of Artificial Sequence Synthetic peptide”
SQSTHFPRT

SEQ ID NO: 11 - protein, 16 aa, Artificial Sequence
  source: /note=“Description of Artificial Sequence Synthetic peptide”
RSSQSLKSSKGNTYLE

SEQ ID NO: 12 - protein, 16 aa, Artificial Sequence
  source: /note=“Description of Artificial Sequence Synthetic peptide”
RSSQSLVSSKGNTYLH

SEQ ID NO: 13 - protein, 7 aa, Artificial Sequence
  source: /note=“Description of Artificial Sequence Synthetic peptide”
KLSNRFS

SEQ ID NO: 14 - protein, 7 aa, Artificial Sequence
  source: /note=“Description of Artificial Sequence Synthetic peptide”
KLSNRFS

SEQ ID NO: 15 - protein, 7 aa, Artificial Sequence
  source: /note=“Description of Artificial Sequence Synthetic peptide”
SQGSHFP

SEQ ID NO: 16 - protein, 7 aa, Artificial Sequence
  source: /note=“Description of Artificial Sequence Synthetic peptide”
SQGSHFP

SEQ ID NO: 17 - protein, 125 aa, Artificial Sequence
  source: /note=“Description of Artificial Sequence Synthetic polypeptide”
EVQLVESGGGLVQPGGSLRLSCAASGFTFSRYAMSWVRQAPGKGLEWVAEISSGGSYPYYPDTVTGRFTISRDNAKNSLYLQMNSLRAEDTAVYYCARVLYYDYDGDRIEVMDYWGQGTLVTVSS

SEQ ID NO: 18 - protein, 112 aa, Artificial Sequence
  source: /note=“Description of Artificial Sequence Synthetic polypeptide”
DVVMTQSPLSLPVTPGEPASISCRSSQSLVSSKGNTYLHWYLQKPGQSPQFLIYKVSNRFSGVPDRFSGSGSGTDFTLKISRVEAEDVGVYFCSQSTHFPRTFGGGTKVEIK

SEQ ID NO: 19 - protein, 112 aa, Artificial Sequence
  source: /note=“Description of Artificial Sequence Synthetic polypeptide”
DVVMTQSPLSLPVTPGEPASISCRSSQSLVSSKGNTYLHWYLQKPGQSPQLLIYKVSNRFSGVPDRFSGSGSGTDFTLKISRVEAEDVGVYYCSQSTHFPRTFGGGTKVEIK

SEQ ID NO: 20 - protein, 454 aa, Artificial Sequence
  source: /note=“Description of Artificial Sequence Synthetic polypeptide”
EVQLVESGGGLVQPGGSLRLSCAASGFTFSRYAMSWVRQAPGKGLEWVAEISSGGSYPYYPDTVTGRFTISRDNAKNSLYLQMNSLRAEDTAVYYCARVLYYDYDGDRIEVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 21 - protein, 219 aa, Artificial Sequence
  source: /note=“Description of Artificial Sequence Synthetic polypeptide”
DVVMTQSPLSLPVTPGEPASISCRSSQSLVSSKGNTYLHWYLQKPGQSPQFLIYKVSNRFSGVPDRFSGSGSGTDFTLKISRVEAEDVGVYFCSQSTHFPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 219 aa, Artificial Sequence
  source: /note=“Description of Artificial Sequence Synthetic polypeptide”
DVVMTQSPLSLPVTPGEPASISCRSSQSLVSSKGNTYLHWYLQKPGQSPQLLIYKVSNRFSGVPDRFSGSGSGTDFTLKISRVEAEDVGVYYCSQSTHFPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC